(19) United States
(12) Patent Application Publication
(10) Pub. No.: US 2004/0030250 A1
(43) Pub. Date: Feb. 12, 2004

(54) Injection system for gene delivery

(76) Inventor: Duncan Stewart, Toronto (CA)

Correspondence Address: NIXON & VANDERHYE, PC, 1100 N GLEBE ROAD, 8TH FLOOR, ARLINGTON, VA, 22201-4714, US

(21) Appl. No.: 10/311,808

(22) PCT Filed: Jun. 22, 2001
(86) PCT No.: PCT/CA01/00953

(30) Foreign Application Priority Data
Jun. 22, 2000 (CA) 2,312,142

(51) Int. Cl. A61B 8/12; A61N 7/00

(52) U.S. Cl. 600/439; 604/22; 600/458; 600/461

(57) ABSTRACT

An injection catheter is provided with a piezoelectric ultrasound emitting device atits distal end, capable of emitting ultrasound of different energy levels. It can be used to image the location of penetration of an internal organ of a patient by the catheter needle, by supply of echocontrast material through the needle and analysis of the ultrasound reflections therefrom. It can also be used to cause tissue perturbation or disruption at the imaged location, e.g. to initiate tissue angiogenesis, by choice of suitable ultrasound energy level of emission, and for delivery of therapeutic substances such as DNA to the disrupted tissue, for better uptake in gene therapy.

### FIELD OF THE INVENTION

This invention relates to catheters, and to medical diagnostic and therapeutic systems utilizing catheters. More specifically, it relates to methods of diagnosing and treating disorders of internal organs of mammalian patients, and catheter apparatus specifically designed for use in such methods.

### BACKGROUND OF THE INVENTION AND PRIOR ART

Injection catheters are known, for delivery of therapeutic substances to internal body organs, by insertion of the catheter through an artery in the patient's body to the vicinity of the organ which it is desired to treat. For example, injection catheters are known for administering treatment to the heart. Such a catheter has a relatively long, flexible tube equipped at its distal end with an injection needle, and at its proximal end with an operating means to operate the injection needle. The catheter is introduced through a puncture in the patient's artery and advanced, with the injection needle in a retracted position, until the vicinity of the organ to be treated, e.g. the myocardium, is reached by its distal end. Then the operating means, outside the patient's body, is actuated so that the injection needle is made to extend beyond the distal end of the catheter tube and into the organ. A further actuation of the operating means may cause discharge of therapeutic fluid, e.g. from a reservoir thereof contained in the catheter tube, or from a syringe attached to the external port of the needle assembly, to be discharged through the needle and into the organ, at the location of tissue penetration. An example of such a catheter is described and illustrated in U.S. Pat. No. 6,004,295 Langer and Stewart, issued Dec. 21, 1999, the entire disclosure of which is incorporated herein by reference.

One application for injection catheters of the above type is in the delivery of extremely small quantities of therapeutic substances to precise locations of an organ or vessel. This can arise, for example, in treatment of a patient's endocardium with a therapeutic fluid such as a DNA solution, in gene therapy. Localized treatment of the endocardium, or other portions of the heart such as the myocardium, to repair local damage, requires very precise control over the location and delivery of the therapeutic DNA fluid, and knowledge on the part of the operator of the precise location at which the therapeutic fluid delivery is being made.

Mukherjee, Debabrata et. al., “Ten-fold Augmentation of Endothelial Uptake of Vascular Endothelial Growth Factor with Ultrasound After Systemic Administration”, Journal of the American College of Cardiology, Vol.25, No. 6, May 2000, pp1678-86, describe perfluorocarbon-exposed sonicated dextrose albumin (PESDA) and its use as ultrasound contrast microbubbles to enhance the uptake of VEGF by the myocardium. PESDA is a solution of microbubbles containing perfluorocarbon (<6 μm in diameter) enveloped in an albumin shell, and is produced by sonicating a solution of dextrose containing albumin and perfluorocarbon gas. The microbubbles act as an ultrasound reflector, so that on application to the vicinity of the microbubble injection, of ultrasound of an appropriate energy level, a reflection of ultrasound from the microbubbles can be detected e.g. with a transducer, and the reflection analyzed to determine the location and distribution of the microbubbles. At higher acoustic energies, the microbubbles burst in situ, and release their contents to their environment.

### SUMMARY OF THE INVENTION

The present invention, from one aspect, provides a catheter having a catheter tube and equipped with means for delivering echocontrast medium and, at its distal end, not only with an injection needle but also with a piezoelectric ultrasound device, capable of emitting ultrasound at two or more energy levels. Other aspects of the invention are various processes, diagnostic and therapeutic, in which such a catheter may be used. The catheter can be introduced into the patient's body e.g. advanced through an artery, to abut the internal organ to be treated, e.g. to abut the myocardium. Then, with the injection needle either adjacent to or extending into the tissue of the organ, low energy ultrasound is delivered to the tissue by the ultrasound crystal. An ultrasound contrast agent such as PESDA is delivered to the tissue by the needle. The low energy ultrasound is reflected and imaged by use of an appropriate transducer, so that the exact location of the injection needle's penetration can be determined by the operator. Subsequently, e.g. when the location of penetration has been verified, the ultrasound energy is raised to a second level, at which it causes focal tissue perturbation or even disruption. This can, for example, be focal myocardial disruption so as to stimulate angiogenesis at the location (e.g. direct myocardial revascularization). As another example, it may be used to ablate conduction tissue during an electrophysiology procedure to lock conduction in an accessory pathway.

Thus according to a first aspect of the invention, there is provided an injection catheter system comprising:

an extended flexible catheter tube for insertion and extension along a patient's artery, said tube having a distal end and a proximal end;

an injection needle at the distal end of the catheter tube capable of being extended beyond the distal end of the catheter tube;

a piezoelectric ultrasound emitting device at the distal end of the catheter tube, said device being capable of emitting ultrasound at a first, lower energy for detection of reflections thereof, and at a second, higher energy for localized disruption of adjacent tissue;

means for delivering ultrasound contrast material through the injection needle;

and means for analyzing reflections of the ultrasound emitted by the ultrasound emitting device and reflected by the ultrasound contrast material.

According to another aspect of the invention, there is provided a process for the diagnosis and/or treatment of localized internal body organ disorders in a mammalian patient, which comprises:

introducing a catheter into the vicinity of the internal body organ surface so that the distal end thereof is adjacent to the surface of the organ;

projecting an injection needle from the distal end of the catheter to penetrate the organ surface;

delivering ultrasound contrast material through the injection needle into the organ surface at the location of penetration;

transmitting ultrasound signals of a first, energy from the distal end of the catheter to the location of penetration of the organ surface and collecting reflected ultrasound signals from said ultrasound contrast material;

analyzing said reflected signals to determine the precise location of penetration of the organ surface by the injection needle;

and transmitting ultrasound signals of a second, tissue-perturbing energy level from the distal end of the catheter following verification of the location of penetration.

### DESCRIPTION OF THE PREFERRED EMBODIMENTS

In addition, in a further embodiment in which a catheter as defined above may, if desired, be used, the invention provides a treatment process whereby a therapeutic substance such as DNA is delivered along with the ultrasound contrast material. The ultrasound, at the same or at a different energy level, causes perturbation, possibly disruption, of the tissue to promote action of the therapeutic substance on the tissue and perhaps to separate it from the contrast material, but at the same time allows the operator to visualize the therapeutic biological and the contrast material as it enters the tissue. Accordingly its location within the tissue can be confirmed. This is a major advantage, especially when treating the myocardium, for example, since it allows the operator to know that indeed intramyocardial agent delivery was accomplished, a difficult determination with other myocardial injection procedures and apparatus. This significantly reduces the risk that injectate might leak back, or even be delivered directly into the circulation.

Another embodiment of the invention contemplates the delivery in this manner of echo contrast material with or without a tissue-affecting substance to the location of desired tissue perturbation or disruption. Once the correct location of the contrast material has been confirmed by ultrasonic imaging, a graduated increase in ultrasound energy can be delivered to cause a focal disruption of tissue at carefully predetermined locations of a body organ or vessel such as the heart. Energy levels can be chosen to result in reversible damage, for example to an accessory electrical pathway, to confirm that a desired therapeutic result can be achieved, and then permanent ablation of the offending tissue can be accomplished with high energy ultrasound.

Another embodiment of the invention, in which the device defined above can also, if desired, be used, combines the benefits of therapeutic substance delivery in combination with echocontrast material, allowing visualization of the focal delivery of the therapeutic material as described above, with the benefits of focal tissue perturbation by ultrasound emission. Once the location of penetration of the organ by the injection needle has been verified by analysis of the ultrasound reflections at the first, lower energy level, the ultrasound energy level from the ultrasound emitting device can be adjusted if necessary to a second level at which it disrupts any combination of the therapeutic material and the echocontrast material, and then adjusted again, if necessary, to raise it to a level at which it causes focal tissue disruption. In this way, the therapeutic substance is delivered to the tissue and transferred to the myocardium in the precise location required to be treated. The ultrasound and the penetration of the injection needle combine to render the tissue and cells at the treatment location physically more receptive to accept the therapeutic substance, e.g. by tissue perturbation or even tissue disruption, for a gene therapy process of enhanced efficiency, and at the same time augment the angiogenic response by eliciting a trigger mechanism for angiogenesis, e.g. tissue injury.

Another preferred application of the catheter and process of the invention is in the diagnosis and treatment of vascular disorders such as stenosis, for example in combination with balloon angioplasty. The delivery of echocontrast material and the imaging of ultrasound reflections into the precise location can be accomplished using modifications of angioplasty balloon catheters to incorporate the ability to inject this material directly into the media of the arterial vesel. As before, the localization of the echo contrast material can be confirmed using standard intravascular ultrasound imaging approaches. Perturbation or even disruption of the tissue at that location can be achieved by the delivery of ultrasound of an appropriate energy level can be used to assist in the repair of the damage. Therapeutic material to counteract tendency to restenosis may be administered to the tissue along with this perturbation-causing ultrasound, which can result in increased gene transfer efficiency as described above.

The preferred echocontrast material is the aforementioned PESDA in microbubble form, although it is by no means limited thereto. Other ultrasound echocontrast materials used for internal imaging in medical applications may be used as well. When a microbubble form of echocontast material is used, the therapeutic material is preferably delivered while enclosed within the microbubbles. The ultrasound, at a higher energy level, causes disruption of the microbubbles to release the therapeutic material, at the precise, accurately visualized delivery location. The disruption of the microbubbles by the ultrasound may cause transient perturbation of myocyte cell membranes, when the process is, as is preferred, applied to treatment of the myocardium with gene therapy, opening pores and allowing genetic material to enter the cells. This may result in increased transfection efficiency.

The piezoelectric ultrasound emitting device which is used in the process and apparatus of the invention is suitably one more piezoelectric crystals, e.g. arranged in an array. The same or different ones of the crystals may both emit ultrasound and receive the reflected ultrasound. Different crystals may be used to transmit the ultrasound of different energy levels, or a single crystal my be arranged to emit a variable ultrasound energy level. The ultrasound emitting and receiving crystal(s) are connected to a stand ultrasound machine for analsis of reflected signals and supply of apptopriate power.

### BRIEF REFERENCE TO THE DRAWINGS

FIG. 1 of the accompanying drawings is a diagrammatic illustration, with parts cut away, of a form of catheter according to the present invention, and useful in the processes of the invention;

FIGS. 2, 3 and 4 are diagrammatic illustrations of the operation of the distal end of a catheter as generally illustrated in FIG. 1, in conducting a process according to a preferred embodiment of the present invention.

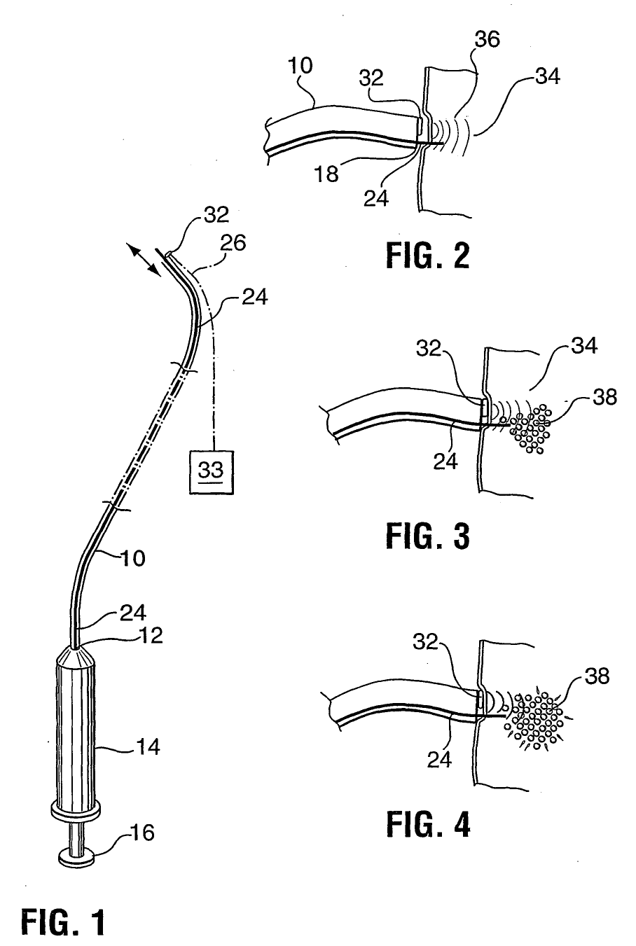

One form of catheter for use in a system according to the invention is diagrammatically illustrated in FIG. 1 of the accompanying drawings. It comprises an elongated flexible catheter tube 10 having at its proximal end 12 a syringe 14 with a plunger 16. A “nitinol” type long injection needle 24, in fluid communication with the syringe 14, extends the length of the catheter 10.

A piezoelectric ultrasound emitting and receiving device 32 is provided at the distal end of the catheter tube 10. The device 32 may comprise a plurality or array of piezoelectric crystals, of known type, and is connected via connector 26 to a standard ultrasound machine 33 for supply of power and for reception and analysis of reflected ultrasound signals.

In operation to treat the myocardium of a patient, the catheter 10 is moved within the artery to the position shown diagrammatically in FIG. 2, with its distal end against the myocardial wall 34 of the patient, and the injection needle 24 extending beyond the distal end of the catheter 10 to penetrate the myocardial wall 34. This causes some degree of disturbance and perturbation of the tissue of the myocardial wall, as indicated at 36.

Next, the syringe 14, 16 on the end of the catheter, is operated so that microbubbles 38 of ultrasound contrast material containing therapeutic DNA are delivered from the syringe 14 and discharged from the injection needle 24 into the tissue of the myocardial wall 34. This is the position shown in FIG. 3 of the accompanying drawings. Ultrasound is now emitted, at a first energy level, from piezoelectric device 32. The frequency of the ultrasound may be adjusted to improve the image received—higher frequencies tend to give shallow penetrations of the ultrasound (which is all that is normally required in the process of the present invention). The reflections of the ultrasound are detected and analyzed, by ultrasound machine 33, to creat an image and to determine the exact location of penetration of the injection needle 24 into the myocardial wall 34.

When this location has been verified, the power of the ultrasound emitted by the piezoelectric device 32 is increased, as indicated in FIG. 4, so that the microbubbles 38 are disrupted and release their therapeutic DNA contents, to the location of penetration. This increased ultrasound power also causes additional tissue and cell perturbation and disruption of the location, for easier acceptance of the DNA material therein, and for triggering angiogenesis in the myocardium at the location of treatment.

The process and apparatus according to the invention provides a means not only for accurate location and positioning of an injection catheter for delivery of therapeutics such as DNA material in gene therapy, but also a process and means for enhancing the uptake of the therapeutic material, by ultrasound perturbation or disruption of cells and tissues at the location to be treated. Very small amounts of therapeutic material, e.g. volumes of the order of 100 microliters can be delivered this way. The material is both visualized and delivered in an advantageous manner by the process and apparatus of the present invention. The process and apparatus allows the operator to know exactly where the gene delivery is taking place, to improve the gene transfer process, and to verify that the delivery and transfer has taken place.

It will be understood that the apparatus and process described herein is by way of example only, and that variations of the apparatus and technique can be made within the scope of the present invention. It is of general application to diagnosis and treatment of internal body organs, vessels and the like, where precise knowledge of the location to be treated is required to be established, and where precise control of the internal location of tissue perturbation or disruption, for initation of angiogenesis, subsequent delivery of therapeutics such as gene therapy, or subsequent application during electrophysiology to produce ablation of electrical or the like is to be undertaken.

1. An injection catheter system comprising:
an extended flexible catheter tube for insertion and extension along a patient's artery, said tube having a distal end and a proximal end;
an injection needle at the distal end of the catheter tube capable of being extended beyond the distal end of the catheter tube;
a piezoelectric ultrasound emitting device at the distal end of the catheter tube;
means for delivering ultrasound contrast material through the injection needle;
and means for analyzing reflections of the ultrasound emitted by the ultrasound emitting device and reflected by the ultrasound contrast material;
characterized in that the piezoelectric ultrasound emitting device is capable of emitting ultrasound at a first, low energy for detection of reflections thereof, and at a second, higher energy for localized perturbation or disruption of adjacent tissue.

2. An injection catheter system according to claim 1, further characterized in that the piezoelectric ultrasound emitting device comprises of plurality of piezoelectric crystals, different crystals thereof being utilized to transmit ultrasound of said first, lower energy and to transmit ultrasound of said second, higher energy.

3 An injection catheter system according to claim 1, further characterized in that the piezoelectric ultrasound emitting device includes a single piezoelectric crystal to transmit said first lower energy and said second higher energy.

4. Use in the assembly of an injection catheter system for treatment of localized internal body organ disorders in an mammalian patient, and comprising an extended flexible catheter tube for insertion and extension along a patient artery,
said tube having a distal end and an proximal end;
an injection needle at the distal end of the catheter tube capable of being extended beyond the distal end of the catheter tube;
means for delivering ultrasound contract material through the injection needle;
and means for analyzing reflections of the ultrasound emitted by the ultrasound emitting device and reflected by the ultrasound contrast material;
of a piezoelectric sound emitting device for mounting at the distal end of the catheter tube, said device being capable of emitting ultrasound at a first, lower energy for detection of reflections thereof, and at a second, higher energy for localized perturbation or disruption of adjacent tissue.

5. A process for the diagnosis and/or treatment of localized internal body organ disorders in a mammalian patient, which comprises:
introducing a catheter into the vicinity of the internal body organ surface so that the distal end thereof is adjacent to the surface of the organ;
projecting an injection needle from the distal end of the catheter to penetrate the organ surface;
delivering ultrasound contrast material through the injection needle into the organ surface at the location of penetration;
and collecting reflected ultrasound signals from said ultrasound contrast material;
characterized in that ultrasound signals of a first energy are transmitted from the distal end of the catheter to the location of penetration of the organ surface and reflected ultrasound signals are collected from said ultrasound contrast material, then said reflected signals are analyzed to determine the precise location of penetration of the organ surface by the injection needle, and then ultrasound signal of a second, tissue-perturbing energy level are transmitted from the distal end of the catheter following verification of the location of penetration.

6. The process of claim 6 further characterized in that the delivered echocontrast material is delivered in association with therapeutic substance.

7. The process of claim 5 or claim 6 further characterized in that the echocontrast material is in the form of microbubbles.

8. The process of claim 6 or claim 7 further characterized in that the echocontrast material is in the form of microbubbles enveloping said therapeutic substance.

9. The process of any of claims 5-8 further characterized in that the therapeutic substance comprises DNA.
